(19) United States
(12) Patent Application Publication
(10) Pub. No.: US 2020/0178934 A1
(43) Pub. Date: Jun. 11, 2020

(54) ULTRASOUND IMAGING SYSTEM AND METHOD FOR DISPLAYING A TARGET OBJECT QUALITY LEVEL

(71) Applicant: General Electric Company, Schenectady, NY (US)

(72) Inventor: Christian Fritz Perrey, Mondsee (AT)

(21) Appl. No.: 16/215,126

(22) Filed: Dec. 10, 2018

(51) Int. Cl. A61B 8/08; A61B 8/00

(52) U.S. Cl. CPC A61B 8/481; A61B 8/54; A61B 8/483; A61B 8/4254; A61B 8/466; A61B 8/5246; A61B 8/5207

(57) ABSTRACT

A method of ultrasound imaging that includes acquiring ultrasound data, and acquiring a target object quality parameter for a target object during the process of acquiring the ultrasound data. One or more processors determine a target object quality level for the target object based on the target object quality parameter and automatically selects a target object quality indicator based on the target object quality level. The one or more processors also generate an image based on the ultrasound data and including the target object quality indicator associated with the target object, and displays the image on a display device.

### FIELD

The subject matter described herein generally relates to an automated ultrasound imaging system and method

### BACKGROUND OF THE INVENTION

Ultrasound imaging procedures oftentimes are used to acquire quantitative or qualitative information from a scanned area related to target objects within the scanned area. An ultrasound imaging system may automatically identify target object parameters such as a length or diameter of an anatomical structure, a volume of blood or fluid flowing through a region in a period of time, a velocity, an average velocity, or a peak velocity acquired from a region of interest of a patient without assistance from a clinician. Still, when acquiring target object parameters from an image, it is important for the ultrasound clinician to know that the acquisition quality was acceptable during the acquisition of the ultrasound data.

Specifically, automated detection and/or segmentation of target objects in ultrasound images helps the user to perform exams more efficiently and may reduce observer variability. However, the automation is not 100% reliable and a clinician still must review the outcome of the automated detection/segmentation to correct the results in case of failures. This review step may be cumbersome, especially in the presence of multiple target objects. For example, when examining follicles in an ovary, multiple target objects are presented, with a clinician required to rereview the automated detection for each target object. This process is both tedious and inefficient, minimizing advantages related to the use of an automated ultrasound device.

### BRIEF DESCRIPTION

The above-mentioned shortcomings, disadvantages and problems are addressed herein which will be understood by reading and understanding the following specification.

In one or more embodiments, a method of ultrasound imaging is provided that includes acquiring ultrasound data and a target object quality parameter for a target object during the process of acquiring the ultrasound data. The method also includes determining, with one or more processors, a target object quality level for the target object based on the target object quality parameter, and automatically selecting a target object quality indicator based on the target object quality level. The method also includes generating an image based on the ultrasound data and including the target object quality indicator associated with the target object, and displaying the image on a display device.

In one or more embodiments, an ultrasound imaging system is provided that includes a probe, a display device, and one or more processors in electronic communication with the probe and the display device. The one or more processors are configured to control the probe to acquire ultrasound data, acquire a target object quality parameter during the process of acquiring the ultrasound data, and determine a target object quality level based on the target object quality parameter. The one or more processors are also configured to select a target object quality indicator associated with the target object and based on the target object quality level, and display an image on the display device that associates the target object quality indicator with the target object based on the ultrasound data.

In one or more embodiments, a non-transitory computer readable medium having stored thereon, a computer program having at least one code section, is provided and said at least one code section being executable by a machine for causing said machine to perform one or more steps including acquiring ultrasound data, and acquiring a first target object quality parameter and a second target object quality parameter from segmented images during the process of acquiring the ultrasound data. The machine also performs the steps of determining, with one or more processors, a first target object quality level based on the first target object quality parameter, and a second target object quality level based on the second target object quality parameter, automatically selecting a first opacity for a first target object based on the first target object quality level and a second opacity for a second target object based on the second target object quality level quality level, and combining the segmented images to form a displayed image having the first target object that is the first opacity and having the second target object that is a second opacity. Optionally, the segmented images are received from a 3-D ultrasound system.

### BRIEF DESCRIPTION OF THE DRAWINGS

FIG. 1 illustrates a schematic diagram of an ultrasound imaging system in accordance with an embodiment.

FIG. 2 is a flow chart of a method for ultrasound imaging in accordance with an embodiment.

FIG. 3 is a schematic representation of an image in accordance with an embodiment.

FIG. 4 is a schematic representation of the image of FIG. 3 from a different view in accordance with an embodiment.

FIG. 5 is a schematic representation of the image of FIG. 3 from a different view in accordance with an embodiment.

FIG. 6 is a schematic representation of a three-dimensional image formed from the images of FIGS. 3-5 in accordance with an embodiment.

### DETAILED DESCRIPTION

The foregoing summary, as well as the following detailed description of various embodiments, will be better understood when read in conjunction with the appended drawings. To the extent that the figures illustrate diagrams of the functional blocks of the various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

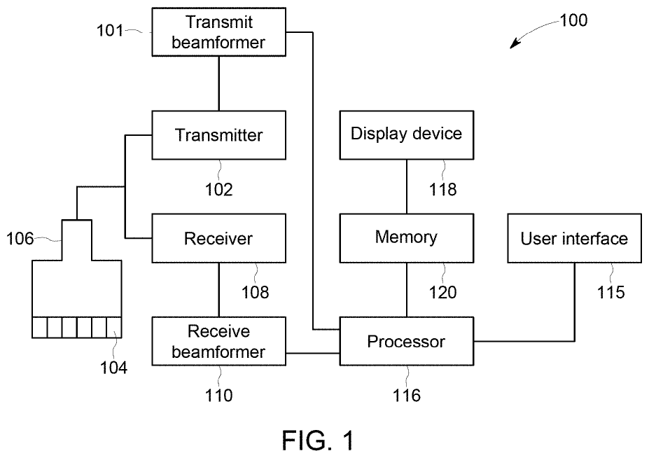

FIG. 1 is a schematic diagram of an ultrasound imaging system 100 in accordance with an embodiment. The ultrasound imaging system 100 includes a transmit beamformer 101 and a transmitter 102 that drive elements 104 within a probe 106 to emit pulsed ultrasonic signals into a body (not shown). The probe 106 may be any type of probe, including a linear probe, a curved array probe, a 1.25D array, a 1.5D array, a 1.75D array, or 2D array probe according to various embodiments. The probe 106 also may be a mechanical probe, such as a mechanical 4D probe or a hybrid probe according to other embodiments. The probe 106 may be used to acquire 4D ultrasound data that contains information about how a volume changes over time. Each of the volumes may include a plurality of 2D images or slices. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104 and the electrical signals are received by a receiver 108.

The electrical signals representing the received echoes are passed through a receive beamformer 110 that outputs ultrasound data. According to some embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the probe 106. The terms “scan” or “scanning” also may refer to acquiring data through the process of transmitting and receiving ultrasonic signals. The terms “data” and “ultrasound data” may refer to either one or more datasets acquired with an ultrasound imaging system. A user interface 115 may be used to control operation of the ultrasound imaging system 100. The user interface may be used to control the input of patient data, or to select various modes, operations, and parameters, and the like. The user interface 115 may include one or more user input devices such as a keyboard, hard keys, a touch pad, a touch screen, a track ball, rotary controls, sliders, soft keys, or any other user input devices.

The ultrasound imaging system 100 also includes a processor 116 to control the transmit beamformer 101, the transmitter 102, the receiver 108 and the receive beamformer 110. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations.

The beamformer 110 may be configured to perform conventional beamforming techniques as well as techniques such as retrospective transmit beamforming (RTB). The processor 116 may control the probe 106 to acquire ultrasound data. The processor 116 controls which of the elements 104 are active and the shape of a beam emitted from the probe 106. The processor 116 is also in electronic communication with a display device 118, and the processor 116 may process the ultrasound data into images for display on the display device 118. For purposes of this disclosure, the term “electronic communication” may be defined to include both wired and wireless connections.

The processor 116 may also include a central processing unit (CPU) according to an embodiment. According to other embodiments, the processor 116 may include other electronic components capable of carrying out processing functions, such as a digital signal processor, a field programmable gate array (FPGA), a graphics processing unit (GPU) or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU).

According to another embodiment, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time frame or volume rates may vary based on the size of the region or volume from which data is acquired and the specific parameters used during the acquisition. The data may be stored temporarily in a buffer (not shown) during a scanning session and processed in less than real-time in a live or off-line operation.

Some embodiments may include multiple processors (not shown) to handle the processing tasks. For example, a first processor may be utilized to demodulate and decimate the RF signal while a second processor may be used to further process the data prior to display as an image. It should be appreciated that other embodiments may use a different arrangement of processors. For embodiments where the receive beamformer 110 is a software beamformer, the processing functions attributed to the processor 116 and the software beamformer hereinabove may be performed by a single processor such as the receive beamformer 110 or the processor 116. Or, the processing functions attributed to the processor 116 and the software beamformer may be allocated in a different manner between any number of separate processing components.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending on the size of the volume and the intended application. For example, many applications involve acquiring ultrasound data at a frame rate of 50 Hz. A memory 120 is included for storing processed frames of acquired data. In one embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

Optionally, embodiments may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments, data may be processed by other or different mode-related modules by the processor 116 (e.g., B-mode, Color Doppler, M-mode, Color M-mode, spectral Doppler, Elastography, TVI, strain, strain rate, and the like) to form 2D or 3D images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from coordinates beam space to display space coordinates. A video processor module may be provided that reads the image frames from a memory and displays the image frames in real time while a procedure is being carried out on a patient. A video processor module may store the image frames in an image memory, from which the images are read and displayed. The ultrasound imaging system 100 may be a console-based system, a laptop, a handheld or hand-carried system, or any other configuration.

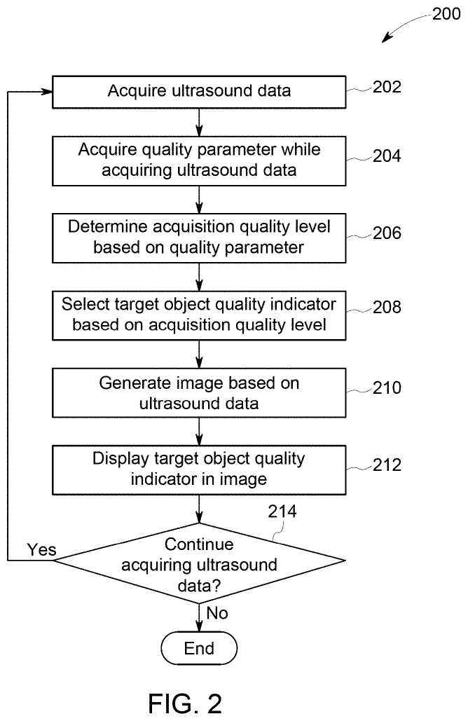

FIG. 2 is a flow chart of a method 200 for ultrasound imaging in accordance with an embodiment. The individual blocks of the flow chart represent operations that may be performed in accordance with the method 200. Additional embodiments may perform the operations shown in a different sequence and/or additional embodiments may include processes not shown in FIG. 2. At least one technical effect of the method 200 is the display of an image generated from ultrasound data that include a plurality of target objects and the display provides color-coding of the target objects, marks the target objects, including with arrows, displays target objects with different opacity, and/or the like to represent the quality or fidelity of the target object image.

FIG. 2 will be described in accordance with an exemplary embodiment where the method 200 is performed by the system 100 shown in FIG. 1. At 202, the processor 116 controls the probe 106 to acquire ultrasound data from a region of a patient. The ultrasound data may include 1D ultrasound data, 2D ultrasound data, 3D ultrasound data or 4D ultrasound data. The ultrasound data may be acquired and displayed in real-time as part of a “live” ultrasound imaging procedure. Or, according to other embodiments, the ultrasound data may be acquired during a first discrete period of time, processed, and then displayed after processing.

At 204, the processor 116 acquires target object quality parameters during the process of acquiring the ultrasound data. Each target object quality parameter may be any parameter that is correlated with the quality of an individual target object in the image. Acquiring the target object quality parameter may include calculating the target object quality parameter from the ultrasound data according to some embodiments, while in other embodiments, acquiring the quality parameter may include acquiring a target object quality parameter based on data that is not ultrasound data. For example, the target object quality parameter may be acquired with a non-ultrasound sensor. The target object quality parameter may, for instance, include a noise level of the image, frame-consistency-over-time metric, a signal intensity, a correctness-of-view metric, a correctness of a flow spectral waveform, or any other parameter associated with object acquisition quality. In general, a lower noise level is correlated with higher target object acquisition quality, a lower amount of probe motion is correlated with higher target object acquisition quality, a higher frame-consistency-over-time is correlated with higher target object acquisition quality, and object size and shape, including roundness is correlated with higher object acquisition quality. The correctness-of-view metric may be calculated by comparing acquired image frames with a standard view using image correlation techniques. Some embodiments may employ deep learning and/or neural networks to determine how closely an acquired image frame matches a standard view.

At 206, the processor 116 determines the acquisition target object quality level based on the target object quality parameter acquired at 204. According to some embodiments, the processor 116 may determine the target object acquisition quality level based on two (2) or more different quality parameters. Or, according to other embodiments, the processor 116 may determine the target object acquisition quality level based on only a single target object quality parameter.

The acquisition target object quality level may, for instance, be determined by a noise level of the image. Specifically, a threshold noise levels may be provided and when the noise level does not exceed any threshold noise level, a first acquisition target object quality level is determined such as having an excellent quality level, while above the first threshold level, yet below a second threshold level of noise level a second acquisition target object quality level is determined such as having an average quality level. Similarly, a noise level the exceeds the second threshold level has a third acquisition target object quality level, such as having a poor quality level.

In yet another example, the acquisition target object quality level is determined based on, or in response to an amount of probe motion. In this example, the change of direction is continually monitored by a sensor, such as an accelerometer to determine the amount of movement of the probe. In this example, the quality level is inversely proportional to the amount of movement and varies over time.

In another example, a frame-consistency-over-time metric, is the target object quality parameter acquired at 204 and an algorithm determines a consistency range. Based on that size of the range or difference of frames over time. Based on the size of the range, or variance between frames, the acquisition target object quality level is determined with a smaller range indicating a higher quality and a larger range indicating a lower quality. Alternatively, an average variance from a mean frame value is utilized, with increased variance indicating a lower quality and decreased variance indicating higher quality. Similarly, an average variance from a median frame value is utilized with increased variance indicating a lower quality. Alternatively, in embodiments deep learning and/or neural networks are utilized to determine the target object quality level.

In another example, signal intensity is used to determine the target object quality level. In one example, a single threshold level is utilized. In the example, intensities above the threshold intensity level are considered a high quality while signals at or below the threshold intensity level are considered low quality.

In yet another example, a correctness-of-view metric is calculated to determine the target object quality level. In one example, a reinforcement learning algorithm is utilized with varying weights provided for different variables depending on the accuracy of reviewed readings. In one example, interference level is one of the variables, while correctness of view metric is another, and signal intensity is yet another. During iterative reviews, the weights are utilized for each variable. Specifically, when a reading is considered accurate during a review, a greater amount of weight is given to a variable reading than if a reading is inaccurate. So, if the interference value is above a threshold value, while the correctness of view metric and the signal intensity value is also below a threshold value, and the reading is determined to be accurate, more weight is placed on the correctness of view threshold value, and signal intensity threshold value and less weight is placed on the interference threshold value. These new weights are then utilized in making a determination whether a next iteration of values result in an accurate reading or determination. Alternatively, the interference threshold value may be increased in response to the accurate reading. Thus, threshold values may also be varied through this iterative process.

In yet another example, a correctness of a flow spectral waveform can be utilized. Again, a reinforcement learning methodology may be utilized. Alternatively, different characteristics such as slope, peak-to-peak height, and the like may be utilized and compared to previous measurements to determine the target object quality level.

In each example, at least one target object quality parameter is acquired and from that target object quality parameter(s) the target object quality level is determined. Thus, additional information related to the target object may be provided to a clinician or user to assist in the review of an image.

Next, at 208, the processor 116 selects a target object quality indicator based on the acquisition target object quality level. In one example embodiment, the target object quality indicator is based on colors. Specifically, the processor 116 may select from at least a first color and a second color, where the second color is different than the first color. According to an embodiment, the first color may represent a first target object acquisition quality level and the second color may represent a second target object acquisition quality level. According to an embodiment, the first color may represent a first range of target object acquisition quality levels and that second color may represent a second range of target object acquisition quality levels, where the second range does not overlap with the first range. The first color may be, for example, green, and the first ranges of acquisition quality levels may represent acquisition target object quality levels that are considered acceptable. The second color may be, for example, red, and the second range of acquisition target object quality levels may represent acquisition quality levels that are unacceptable.

According to other embodiments, the processor 116 may select from more than two colors representing more than two discrete ranges of acquisition quality levels. For example, a first color, such as green, may represent a first acquisition quality level; a second color, such as yellow, may represent a second acquisition quality level; and a third color, such as red, may represent a third acquisition quality level. Or, the first color may represent a first range of acquisition quality levels, the second color may represent a second range of acquisition quality levels, and the third color may represent a third range of acquisition quality levels. The first range of acquisition quality levels, the second range of acquisition quality levels, and the third range of acquisition quality levels may each be discrete, non-overlapping ranges according to an embodiment. According to other embodiments, more than three different colors may be used to represent various acquisition quality levels or various ranges of acquisition quality levels.

According to an embodiment using three colors, green may be the first color and it may be used to represent an acquisition quality level that is high, red may be the second color and it may be used to represent an acquisition quality level that is low, and yellow may be the third color and it may be used to represent an acquisition quality level that is medium (i.e., in between the high acquisition quality level and the low acquisition quality level). The acquisition quality levels (i.e., high, medium and low, according to an embodiment) may be preset on the processor 116 at the factory or they may be user definable. The user may, for instance, assign a range of quality parameter values to each acquisition quality level. Likewise, the user may assign various acquisition quality levels to acquisition quality values or the user may define a range of acquisition quality levels associated with each color.

In an alternative embodiment, the acquisition target object quality levels are expressed on a numeric scale, such as, for example 1-10. In this embodiment, highlighting symbols such as arrows can point to a target object on an image with a number associated with each arrow. Thus, numbers 1-3 can represent a target object with a poor target object quality level that a clinician recognizes as poor target object quality level and will ensure to take a closer, or more detailed look at the target object during review. Similarly, numbers 8-10 can represent an excellent target object quality level. Thus, when a clinician observes a target object with an 8-10 target object quality level, the clinician can more quickly and efficiently scan through these target objects with confidence the image diagnosis by the automated ultrasound device has a high probability of being accurate.

In yet another alternative embodiment, the target objects are presented in different opacities, again with the differing opacities representing different qualities of a diagnosis or reading. Thus, again, based on the opacity of target objects in an image, the target object quality levels are presented to a clinician, providing a clinician with an informed review of an image and efficient use of time in reviewing each target object.

In all, an imaging system includes a processor that determines a target object quality level/parameter/indicator base on target object characteristics such as roundness, size, shape, or the like for each target object. Then the processor highlights or provides a target object quality indictor on target objects in an image at least on target objects having quality below a threshold limit. Consequently, a clinician is able spend more time reviewing target objects of low quality to correct incorrect diagnoses of the automated imaging device.

These target object quality indicators can include presenting the target objects in different opacity, different color, marking target objects with arrows, color arrows, words, numbers, other such indicators, or the like. In example embodiments, optionally, when displaying target objects with different opacity, the quality can be mapped to the grade of opacity. As example the target object quality indicator can be based on a number between one (1) and zero (0), where one (1) is displayed as a first opacity that is solid, and zero (0) is a second opacity that is opaque and almost not visible.

In addition, in one example embodiment, optionally, target objects above a threshold quality level can be temporarily blanked, or removed from the image, so only the cases that potentially need additional manual correction are displayed to improve efficiencies. Specifically, by visualizing and/or marking the target objects that may need correction, the correction process increases in speed, is more efficient, and less cumbersome. This leads to reduced examination times and higher patient throughput.

Next, at 210, the processor 116 generates an image based on the ultrasound data. The image may be a 1D image, a 2D image, a 3D image or a 4D image. The image may be generated from any mode of ultrasound data. For example, the image may be a B-mode image, a Color Doppler image, a M-mode image, a Color M-mode image, a spectral Doppler image, an Elastography image, a TVI image, or any other type of image generated from ultrasound data. The ultrasound data may be acquired, and the image may be displayed in real-time as part of a “live” ultrasound imaging procedure. According to embodiments, the image may be a still frame generated from the ultrasound data. According to other embodiments, the processor 116 may generate images from two or more different imaging modes at 210 based on the ultrasound data. For example, in a VTI mode, the processor 116 may generate both a B-mode image and a spectral Doppler image based on the ultrasound data. In an IVC mode, the processor 116 may generate both a B-mode image and an M-mode image based on the ultrasound data. Then the processor 116 displays the image on the display device 118.

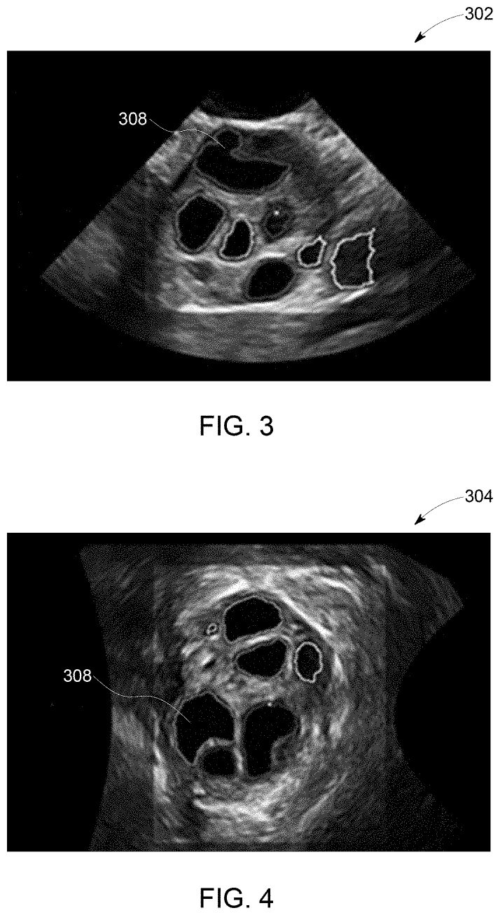

At 212, the processor 116 communicates to a display device to display a target object quality indicator associated with each target object in the image. As described above, the target object quality level may be a color-coded scheme with each color, or shade of color representing a different level of quality. Alternatively, numbers, letters, opacity, arrow indicators, or the like may be used to communicate to a clinician the quality of the target object in the image for the purposes of review of the image and diagnosis by the clinician. Examples of types of information that may be displayed will be described hereinafter with respect to FIGS. 3-6.

At 214, the processor 116 determines if it is desired to continue acquiring ultrasound data. If it is desired to continue acquiring ultrasound data, the method 200 may repeat 202, 204, 206, 208, 210, and 212. According to an embodiment where the ultrasound image is a live image, 202, 204, 206, 208, 210, 212, and 214 may be iteratively repeated one or more times during the acquisition and display of the live image. In an example, 204, 206, 208, 210, 212, and 214 may, for instance, all be performed multiple times during the process of acquiring the ultrasound data at 202.

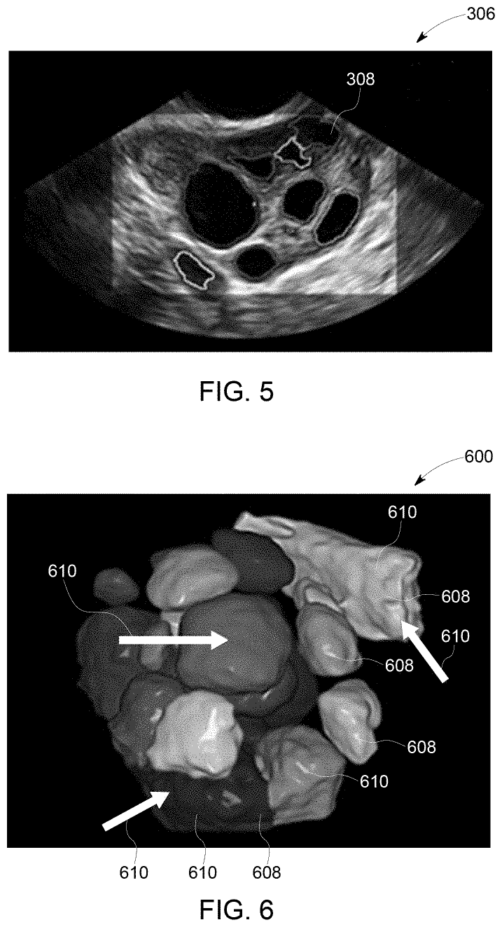

FIG. 3 illustrates a schematic representation of a first ultrasound segmentation image of a patient's anatomy in accordance with an embodiment. FIG. 4 illustrates a schematic representation of a second ultrasound segmentation image of the patient's anatomy of FIG. 3. FIG. 5 illustrates a schematic representation of a third ultrasound segmentation image of the patient's anatomy of FIG. 3.

With reference to FIGS. 3-5, the figures collectively illustrate ultrasound images of the internal anatomy of a patient that is being examined by a clinician, with FIG. 3 representing a first ultrasound segmentation image 302, FIG. 4 representing a second ultrasound segmentation image 304, and FIG. 5 representing a third ultrasound segmentation image 306. In each ultrasound segmentation image 302, 304, and 306 a plurality of target objects 308 are presented with each target object 308 being related to the internal anatomy of the patient. In one example embodiment the ultrasound segmentation images 302, 304, and 306 are of an ovary and the target objects are follicles of the ovary. In another example embodiment the ultrasound segmentation images 302, 304, and 306 are of an unborn baby and the target objects 308 are the heart and lungs of the unborn baby. By analyzing image quality parameters associated with each segmentation image 302, 304, and 306 as described in relation to the method of FIG. 2, a target object quality parameter for each target object 308 is determined. In this manner, a first target object has first target object quality parameters while a second target object has second target quality parameters.

FIG. 6 illustrates a schematic view of a combined image 600 utilizing the first ultrasound segmentation 302, the second ultrasound segmentation 304, and the third ultrasound segmentation 306 of FIGS. 3-5 utilizing the methodology as described in relation to FIG. 2. In the combined image 600 a plurality of target objects 608 are provided with target object quality indicators 610 provided to indicate to the physician the quality of the target objects in the image.

In the example of the FIG. 6, the target object quality indicators 610 are black and white (or opaque) arrows, with the white arrows, or first opacity quality indicators, representing a high-quality level for the pointed to target object 608, while the black arrows, or second opacity quality indicator, represent a low quality level for the pointed to target objects. Specifically, a processor of the imaging system calculates a quality or fidelity parameter for each detected, or segmented, target object 608 and visualizes this quality information by automatically selecting target object quality indicators 610 based on the quality level of the target objects 608 determined based on the target object quality parameters. Consequently, with this information, during the review, a clinician can focus on target objects 608 with a low quality, or fidelity, level and correct them. Additionally, the clinician can spend less time reviewing target objects 608 identified as having a high target object quality level, or high-fidelity rating. This increases the workflow efficiency, reduces user frustration, and improves test reliability.

Thus, in an example embodiment when the target objects 608 are follicles of an ovary a clinician can immediately identify that the target objects 608 pointed to by the white arrow have a high probability of being a follicle, and the target objects 608 pointed to by the black arrow have a low probability of being a follicle. Therefore, detected follicles of the ovary that have a black arrow pointing at them need to be reviewed closely by a clinician to verify those target objects 608 are follicles. While the target object quality indicators 610 are white and black arrows in this exemplary embodiment, in other embodiments color coding, numbers, letters, opacity, combinations thereof, and the like as previously described may similarly be used as target object quality indicators 610 to provide the clinician with an efficient way of reviewing the combined image 600 and ensure that potential problem areas of the image 600 are more closely examined.

As an example, during automated detection and segmentation of stimulated follicles in a 3D acquisition of the ovary, the imaging system processor is configured to find and segment dark cavities in the volumetric data set and automatically select and display a quality indicator 610 related to the dark cavity. In one example embodiment, the dark cavities are illustrated as color coded regions wherein the color coded regions represent the quality indicators 610.

Therefore, in the follicles example, a target object 608 in the combined image 600 that is round has a high probability of being a follicle and is denoted by the white arrow quality indicator 610. Meanwhile, target objects 608 that have an irregular or jagged shape represent potentially false segmentations and therefore indicated with a quality indicator 610 that is a dark arrow to represent the target object 608 should be reviewed more closely by a clinician. Therefore, in this example, the quality indicator 610 is based on a predetermined shape of the target object, with smoother, more rounded object having a higher image quality than irregular or jagged shapes.

Similarly, in an alternative example, the quality indicator 610 automatically selected is the volume of the target object 608. In particular, a target object quality indicator 610 can be based on a threshold volume of the shape of the target object. Thus, even if the shape is rounded, if the shape does not meet a threshold volume, such as in one example one inch, and in another example at least 5% of the display screen, then the image acquisition quality is poor. Similarly, the larger the volume, the greater the object image quality.

In yet another example embodiment, the quality indicator 610 automatically selected is the opacity of the target object. Thus, the target object 608 is displayed in a first opacity, that may be dark, where the dark opacity is the quality indicator 610 of the target object 608. Then another target object 608 is displayed in a second opacity, that may be opaque, where the opaqueness is the quality indicator of the target object. Thus, a clinician understands that darker target objects 608 having the first opacity represent a higher image acquisition quality than the second opacity that is a nearly opaque target object.

Provided is an improved system and method for quickly and efficiently reviewing ultrasound images and results of an automated ultrasound device. A target object quality indictor is utilized to inform a clinician, or user of the quality of target objects in the image. Consequently, high quality target objects may be reviewed more quickly, while a clinician can review poor quality target objects more closely and spend more time reviewing to verify an incorrect reading is not detected. Consequently, the review process becomes more efficient, false readings may be more easily be detected, and clinician confidence in the automated results is enhanced.

Also provided is a method of ultrasound imaging is provided that includes acquiring ultrasound data and a target object quality parameter for a target object during the process of acquiring the ultrasound data. The method also includes determining, with one or more processors, a target object quality level for the target object based on the target object quality parameter, and automatically selecting a target object quality indicator based on the target object quality level. The method also includes generating an image based on the ultrasound data and including the target object quality indicator associated with the target object, and displaying the image on a display device.

Optionally, the method in the method, the target object quality indicator is represented by a color of the target object in the image. Also, optionally, the target object is a first target object, the target object quality indicator is a first target object quality indicator, and the target object quality indicator is a first target object quality indicator. In this example the method also includes acquiring a second target object quality parameter for a second target object during the process of acquiring the ultrasound data, and determining, with the processor, a second target object quality level for the second target object based of the second target object quality parameter. In this example the method also includes automatically selecting a second target object quality indicator based on the second target quality level, and generating the image including the second target object quality indicator associated with the second target object. In this example, the first target object quality indicator is a color of the first target object, and the second target object quality indicator is a color of the second target object. Also, in this method, the color of the first target object is different than the color of the second target obj ect.

Optionally, the target object quality indicator is a number. Also, optionally, the target object quality parameter is a shape of the target object. In such an example, the target object quality indicator is based on a difference between the shape of the target object and a pre-determined shape. Alternatively, in the example embodiment the target object quality indicator is further based on whether the shape of the target object has a volume that is greater than a threshold volume of the pre-determine shape. Optionally, the image is one of a one-dimensional ultrasound image, a two-dimensional ultrasound image; a three-dimensional ultrasound image, or a four-dimension ultrasound image. Also optionally, the target object quality parameter is acquired by analyzing a plurality of segmented ultrasound images.

Also provided is an ultrasound imaging system is provided that includes a probe, a display device, and a processor in electronic communication with the probe and the display device. The processor is configured to control the probe to acquire ultrasound data, acquire a target object quality parameter during the process of acquiring the ultrasound data, and determine a target object quality level based on the target object quality parameter. The processor is also configured to select a target object quality indicator associated with the target object and based on the target object quality level, and display an image on the display device that associates the target object quality indicator with the target object based on the ultrasound data.

Optionally, the target object quality indicator is a color and the target object is displayed in the color to associate the target object quality indicator with the target object. Also, optionally, the processor is further configured to combine segmented image data from the ultrasound data to form the image, and the target object quality parameter is based on the segmented image data. In this example embodiment, the image formed from the combined segmented image data is a rendered image and the quality parameter is a shape of the target object in the rendered image.

Optionally, the target object is a first target object, the target object quality indicator is a first target object quality indicator, and the processor is further configured to display the image on the display device based on the ultrasound data that associates a second target object quality indicator with a second target object. In this example embodiment, the first target object quality indicator and second target object quality indicator are different. Also in this example embodiment, the first target object quality indicator is a first color and the first target object is displayed in the first color to associate the first target object quality indicator with the first target object, and the second target object quality indicator is a second color and the second target object is displayed in the second color to associate the second target object quality indicator with the second target object.

In one or more embodiments, a non-transitory computer readable medium having stored thereon, a computer program having at least one code section, is provided and said at least one code section being executable by a machine for causing said machine to perform one or more steps including acquiring ultrasound data, and acquiring a first target object quality parameter and a second target object quality parameter from segmented images during the process of acquiring the ultrasound data. The machine also performs the steps of determining, with a processor, a first target object quality level based on the first target object quality parameter, and a second target object quality level based on the second target object quality parameter, automatically selecting a first opacity for a first target object based on the first target object quality level and a second opacity for a second target object based on the second target object quality level quality level, and combining the segmented images to form a displayed image having the first target object that is the first opacity and having the second target object that is a second opacity. Optionally, the segmented images are received from a 3-D ultrasound system.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. Dimensions, types of materials, orientations of the various components, and the number and positions of the various components described herein are intended to define parameters of certain embodiments, and are by no means limiting and are merely example embodiments. Many other embodiments and modifications within the spirit and scope of the claims will be apparent to those of ordinary skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the invention, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method of ultrasound imaging comprising:
acquiring ultrasound data;
acquiring a target object quality parameter for a target object during the process of acquiring the ultrasound data;
determining, with one or more processors, a target object quality level for the target object based on the target object quality parameter;
automatically selecting a target object quality indicator based on the target object quality level;
generating an image based on the ultrasound data and including the target object quality indicator associated with the target object; and
displaying the image on a display device.

2. The method of claim 1, wherein the target object quality indicator is represented by a color of the target object in the image.

3. The method of claim 1, wherein the target object is a first target object, the target object quality indicator is a first target object quality indicator, and the target object quality indicator is a first target object quality indicator and further comprising:
acquiring a second target object quality parameter for a second target object during the process of acquiring the ultrasound data;
determining, with the one or more processors, a second target object quality level for the second target object based of the second target object quality parameter;
automatically selecting a second target object quality indicator based on the second target quality level; and
generating the image including the second target object quality indicator associated with the second target object.

4. The method of claim 3, wherein the first target object quality indicator is a color of the first target object, and the second target object quality indicator is a color of the second target object.

5. The method of claim 4, wherein the color of the first target object is different than the color of the second target object.

6. The method of claim 1, wherein the target object quality indicator is a number.

7. The method of claim 1, wherein the target object quality parameter is a shape of the target object

8. The method of claim 7, wherein the target object quality indicator is based on a difference between the shape of the target object and a pre-determined shape.

9. The method of claim 8, wherein the target object quality indicator is further based on a threshold volume of the shape.

10. The method of claim 1, wherein the image is one of a one-dimensional ultrasound image, a two-dimensional ultrasound image; a three-dimensional ultrasound image, or a four-dimension ultrasound image.

11. The method of claim 1, wherein the target object quality parameter is acquired by analyzing a plurality of segmented ultrasound images.

12. An ultrasound imaging system comprising:
a probe;
a display device; and
one or more processors in electronic communication with the probe and the display device, wherein the one or more processors are configured to:
control the probe to acquire ultrasound data;
acquire a target object quality parameter during the process of acquiring the ultrasound data;
determine a target object quality level based on the target object quality parameter;
select a target object quality indicator associated with the target object and based on the target object quality level; and
display an image on the display device that associates the target object quality indicator with the target object based on the acquired ultrasound data.

13. The ultrasound imaging system of claim 12, wherein the target object quality indicator is a color and the target object is displayed in the color to associate the target object quality indicator with the target object.

14. The ultrasound imaging system of claim 12, wherein the one or more processors are further configured to:
combine segmented image data from the ultrasound data to form the image;
wherein the target object quality parameter is based on the segmented image data.

15. The ultrasound imaging system of claim 14, wherein the image formed from the combined segmented image data is a rendered image and the quality parameter is a shape of the target object in the rendered image.

16. The ultrasound imaging system of claim 12, wherein the target object is a first target object, the target object quality indicator is a first target object quality indicator, and the one or more processors is further configured to:
display the image on the display device based on the ultrasound data that associates a second target object quality indicator with a second target object.

17. The ultrasound imaging system of claim 16, wherein the first target object quality indicator and second target object quality indicator are different.

18. The ultrasound imaging system of claim 16, wherein the first target object quality indicator is a first color and the first target object is displayed in the first color to associate the first target object quality indicator with the first target object, and the second target object quality indicator is a second color and the second target object is displayed in the second color to associate the second target object quality indicator with the second target object.

19. A non-transitory computer readable medium having stored thereon, a computer program having at least one code section, said at least one code section being executable by a machine for causing said machine to perform one or more steps comprising:
acquiring ultrasound data;
acquiring a first target object quality parameter and a second target object quality parameter from segmented images during the process of acquiring the ultrasound data;
determining, with one or more processors, a first target object quality level based on the first target object quality parameter, and a second target object quality level based on the second target object quality parameter;
automatically selecting a first opacity for a first target object based on the first target object quality level and a second opacity for a second target object based on the second target object quality level quality level; and
combining the segmented images to form a displayed image having the first target object that is the first opacity and having the second target object that is a second opacity.

20. The non-transitory computer readable medium of claim 19, wherein the segmented images are received from a 3-D ultrasound system.
